(19) United States
(12) Patent Application Publication
(10) Pub. No.: US 2026/0345311 A1
(43) Pub. Date: Nov. 5, 2026

(54) VESSEL PUMPS AND METHODS OF USE

(71) Applicants: Texas Medical Center, Houston, TX (US); Baylor College of Medicine, Houston, TX (US)

(72) Inventors: Sorosh Amiri, Houston, TX (US); William E. Cohn, Bellaire, TX (US); Mohammad Ghasemi Rad, Houston, TX (US); Nicolò Garbin, Houston, TX (US)

(21) Appl. No.: 19/662,916

(22) Filed: Apr. 29, 2026

Related U.S. Application Data
(60) Provisional application No. 63/797,810, filed on Apr. 30, 2025.

(51) Int. Cl. A61F 2/06

(52) U.S. Cl. CPC A61F 2/06; A61F 2220/0025; A61F 2230/0006; A61F 2230/001; A61F 2230/0054; A61F 2230/0069; A61F 2250/0007

(57) ABSTRACT

Provided herein is a vessel pump positionable inside a vessel of a patient. A vessel pump may have a first portion comprising a first inlet fluidically connected to a first fluidic channel, the first inlet comprising an opening allowing a first fluid originating from the vessel of the patient to flow through the opening into the first fluidic channel. A vessel pump may have a second portion comprising a second fluidic channel fluidically connected to an output end. A vessel pump may have a second inlet positioned at a junction between the first portion and the second portion, wherein the second inlet is in fluidic communication with the single fluidic channel through an angled surface that forms an acute angle relative to a longitudinal axis of the first portion.

### CROSS-REFERENCE TO RELATED APPLICATIONS

This application is a U.S. Non Provisional patent application which claims the benefit and priority to U.S. Provisional Patent Application No. 63/797,810 titled “VESSEL PUMPS AND METHODS OF USE” filed on Apr. 30, 2025, which is hereby incorporated by reference herein in its entirety.

### TECHNICAL FIELD

The present disclosure relates to valves. In particular, the present disclosure relates to vessel pumps and methods of use.

### SUMMARY

Provided herein is a vessel pump positionable inside a vessel of a patient. A vessel pump may have a first portion comprising a first inlet fluidically connected to a first fluidic channel, the first inlet comprising an opening allowing a first fluid originating from the vessel of the patient to flow through the opening into the first fluidic channel. A vessel pump may have a second portion comprising a second fluidic channel fluidically connected to an output end. A vessel pump may have a connecting structure connecting the first portion to the second portion, wherein the first fluidic channel and the second fluidic channel form a single fluidic channel when the first portion and the second portion are connected. A vessel pump may have a second inlet positioned at a junction between the first portion and the second portion, wherein the second inlet is in fluidic communication with the single fluidic channel through an angled surface that forms an acute angle relative to a longitudinal axis of the first portion, the second inlet having an opening width that, when the vessel pump is positioned inside the vessel, is sufficient to establish a pressure differential between a second fluid adjacent to the vessel and the first fluid flowing in the single fluidic channel, wherein the pressure differential causes at least a portion of the second fluid to be directed into the single fluidic channel through the second inlet.

Provided herein is a deployable vessel pump. A deployable vessel pump may have a tubular structure comprising a first end, a second end, and a fluidic channel formed therebetween, wherein the first end comprises a first inlet comprising an opening allowing a flow of a first fluid of the vessel into the fluidic channel through the opening. A deployable vessel pump may have a second inlet formed within the tubular structure, wherein the second inlet is in fluidic communication with the fluidic channel through an angled surface that forms an acute angle relative to a longitudinal axis of the tubular structure, the second inlet having an opening width that, when the deployable vessel pump is positioned inside the vessel, is sufficient to establish a pressure differential between a second fluid adjacent to the vessel and the first fluid flowing in the fluidic channel, wherein the pressure differential causes at least a portion of the second fluid to be directed into the fluidic channel through the second inlet. A deployable vessel pump may have a graft circumferentially encompassing the tubular structure, wherein at least a portion of the graft is removed, thereby allowing a fluidic connection between the second fluid and the second inlet, forming a combined fluid.

Provided herein is a method of directing fluid within a patient using a deployable vessel pump. A method may include deploying a graft into a vessel of the patient. A method may include inserting a tool within the graft. A method may include puncturing, by the tool, the graft and a portion of the vessel. A method may include placing the deployable vessel pump into the graft, the deployable vessel pump comprising a first end, a second end, and a fluidic channel form there between, the first end comprising a first inlet, the first inlet comprising an opening allowing a flow of a first fluid originating from the vessel to flow into the fluidic channel through the first inlet. A method may include aligning a second inlet formed within the deployable vessel pump with the hole, wherein the second inlet is in fluidic communication with the fluidic channel through an angled surface that forms an acute angle relative to a longitudinal axis of the tubular structure, the second inlet having an opening width that, when the deployable vessel pump is positioned inside the vessel, is sufficient to establish a pressure differential between a second fluid adjacent to the vessel and the first fluid flowing in the fluidic channel, wherein the pressure differential causes at least a portion of the second fluid to be directed into the fluidic channel through the second inlet. A method may include directing both a first fluid originating from the vessel and a second fluid originating from the fluid proximate the hole of the vessel through the fluidic channel of the vessel pump.

The above and other preferred features, including various details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained herein may be employed in various and numerous embodiments.

### BRIEF DESCRIPTION OF THE DRAWINGS

The disclosed embodiments have advantages and features which will be more readily apparent from the detailed description, the appended claims, and the accompanying figures (or drawings). A brief introduction of the figures is below.

FIG. 1 illustrates a perspective view of a cross-section of a vessel pump;

FIG. 2 illustrates a top down view of the bottom half of the vessel pump of FIG. 1;

FIG. 3 illustrates a side view of the vessel pump of FIG. 1;

FIG. 4 illustrates a front view of the vessel pump of FIG. 1;

FIG. 5 illustrates an exploded view of a deployable vessel pump adjacent a vessel;

FIG. 6 illustrates the deployable vessel pump of FIG. 5 within a vessel;

FIG. 7A illustrates a left side view of a deployable vessel pump;

FIG. 7B illustrates a right side view of a deployable vessel pump;

FIG. 8 illustrates a side view of a deployable vessel pump within a vessel;

FIG. 9 illustrates a side perspective view of a vessel pump;

FIG. 10 illustrates a side x-ray view of the vessel pump of FIG. 10;

FIG. 11 illustrates a front view of the vessel pump of FIG. 10;

FIG. 12 illustrates a side cross-sectional view of a vessel pump;

FIGS. 13A-13B illustrates another side cross-sectional view of a vessel pump;

FIGS. 14A-14B illustrate side perspective views of valves of a vessel pump in closed and open states;

FIG. 15 illustrates a flowchart of a method of directing a flow of fluid within a patient using a deployable vessel pump; and

FIG. 16 illustrates an anatomical representation of a vessel pump within a vessel.

### DETAILED DESCRIPTION

Reference will now be made in detail to several embodiments, examples of which are illustrated in the accompanying figures. The Figures (Figs.) and the following description relate to preferred embodiments by way of illustration only. It is noted that wherever practicable similar or like reference numbers may be use in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

The human body has many vessels that carry various fluids. In some instances, there may be buildup of unwanted fluid surrounding one or more vessels that may cause health issues to an individual. However, removal of unwanted fluid may require intrusive and complicated procedures that may also require external power to pump out unwanted fluid. Embodiments described herein provide for devices and methods that may be minimally invasive and passively powered while reducing fluid buildup in the human body.

### Vessel Pump

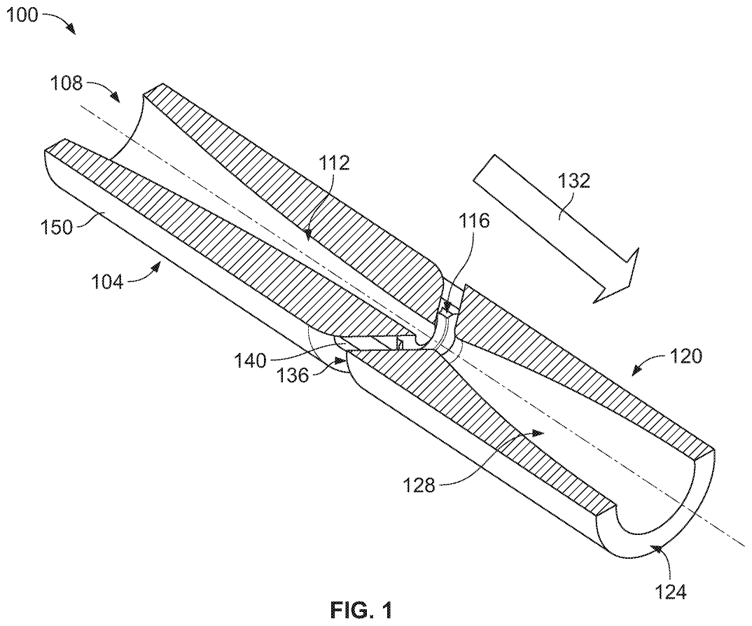

Referring now to FIG. 1, a perspective view of a cross-sectional area of a bottom-half of vessel pump 100 is provided. A “vessel” as used in this disclosure refers to substantially cylindrically shaped structures within the human body that is capable of allowing a flow of fluid therein. Vessels may be, but are not limited to, veins, arteries, or other structures that may be found within the human body. Vessel pump 100 may be insertable into one or more veins, arteries, or any other vessel of an individual, as described herein. Veins may include, but are not limited to, deep veins, pulmonary veins, systemic veins, portal veins, communicating veins, perforating veins, and/or any other type of veins. Veins may be located in an abdomen, chest, back, spinal column, limbs, head, or any other part of the human body. A “valve” as used in this disclosure refers to a device capable of directing a flow of fluid in a direction. Vessel pump 100 may act as, but is not limited to, a one-way valve, as described in more detail herein.

Vessel pump 100 may include first portion 104. A “portion” as used in this disclosure refers to a part of an object. In various embodiments, a portion of the vessel pump 100 may be removably coupled to one or more other portions of the vessel pump 100. In various embodiments, a portion of the vessel pump 100 is not removably coupled to one or more other portions of the vessel pump 100 (e.g., the portion of the vessel pump 100 is fused to one or more other portions of the vessel pump 100). For instance vessel pump 100 may be formed as a tubular structure including first portion 104, second portion 120, and connecting structure 116 mechanically connecting first portion 104 to second portion 120. “Mechanically connecting” or “mechanical communication” as used herein refers to any form of physical attachment between two or more objects. Connections between first portion 104 and second portion 120 via connecting structure 116 are described in further detail herein.

Referring still to FIG. 1, first portion 104 may be cylindrically shaped. For instance, first portion 104 may be shaped to fit inside a vessel of an individual. “Substantially cylindrically shaped” refers to a geometry of an object that has a radius that varies within about 15% of a value circumferentially around the object. For instance, some vessels within the human body may not be perfectly cylindrically shaped, and may have slightly different radii circumferentially around them. First portion 104 may be substantially cylindrically shaped which may match a geometry of one or more vessels of an individual, allowing for placement of first portion 104 inside a vessel of an individual. First portion 104 may be oriented within a vessel to be facing a direction of flow of fluid within the vessel of an individual, such as direction 132. For instance, first portion 104 may receive fluid as fluid moves in direction 132. First portion 104 may include first inlet 108. An “inlet” as used in this disclosure refers to an opening capable of allowing a flow of fluid to enter therein. First inlet 108 may be shaped cylindrically or substantially cylindrically. First inlet 108 may be designed to allow a flow of fluid from within a vessel of an individual to enter within first fluidic channel 112. A “fluidic channel” as used in this disclosure refers to a structure capable of providing a path for fluid to travel within. First inlet 108 may be fluidically connected to first fluidic channel 112. “Fluidically connected” as used in this disclosure refers to a communication between two or ore objects that allows a transfer of fluid therebetween. Fluid may travel in direction 132 from first inlet 108 into first fluidic channel 112. First fluidic channel 112 may direct a flow of fluid from first inlet 108 towards second fluid channel 128.

A “fluidic channel” as used in this disclosure is any pathway capable of allowing a flow of fluid therein. First inlet 108 may be positioned at a proximal end of vessel pump 100. A proximal end of vessel pump 100 may be an end of vessel pump 100 facing a flow of fluid within a vessel. For instance, a proximal end of vessel pump 100 may receive fluid as fluid moves in direction 132. First inlet 108 may be located at a proximal end of vessel pump 100, facing towards a flow of fluid within a vessel flowing in direction 132. A flow of fluid in direction 132 may occur through one or more bio-hydrodynamic processes. A “bio-hydrodynamic process” as used in this disclosure refers to an operation of pumping a fluid through a vessel by one or more muscles of the human body. For instance, the cardiac muscle may pump blood through one or more veins of the human body. Vessel pump 100 may be positioned within a vein or an artery of the human body, which may allow for a flow of fluid in direction 132 to occur through first fluidic channel 112. Fluid may include, but is not limited to, blood, cerebral spinal fluid, bile, lymphatic fluid, digestive fluid, urine, or any other fluid that may be found within the human body.

In some embodiments, vessel pump 100 may include second inlet 116. Second inlet 116 may be a gap between first portion 104 and second portion 120. In some embodiments, second inlet 116 may be positioned at a junction where the first portion 104 and second portion 120 meet. The size and dimension of the junction where the first portion 104 and the second portion 120 may be defined by one or more connecting structures 136. For instance, connecting structure 136 may include one or more legs 140 that may connect first portion 104 to second portion 120. In some embodiments, connecting structure 136 may include at least two or more legs 140 or at least three or more legs 140. A “leg” as used in this disclosure is a protruding structure that is designed to mate to and/or contact an object. Legs 140 of connecting structure 136 may be cylindrical, semi-circular, rectangular, square, helical, or other shapes. In some embodiments, legs 140 of connecting structure 136 may be adjustable. For instance, legs 140 may be screw-like which may allow for an increase or decrease in length of one or more legs 140 relative to first portion 104 and/or second portion 120 upon a rotation of one or more legs 140 in a clockwise or counter clockwise direction. In some embodiments, individual legs of legs 140 may be adjustable in length. In some embodiments, connecting structure 136 may have a first leg positioned on a left side of vessel pump 100, a second leg positioned on a right side of vessel pump 100, and a third leg positioned at a top of vessel pump 100. A three-leg formation may allow for a prevention of first portion 104 and/or second portion 120 to become displaced in an X-axis, Y-axis, and/or Z-axis through providing a normal force opposing a force in an X-axis, Y-axis, and/or Z-axis direction.

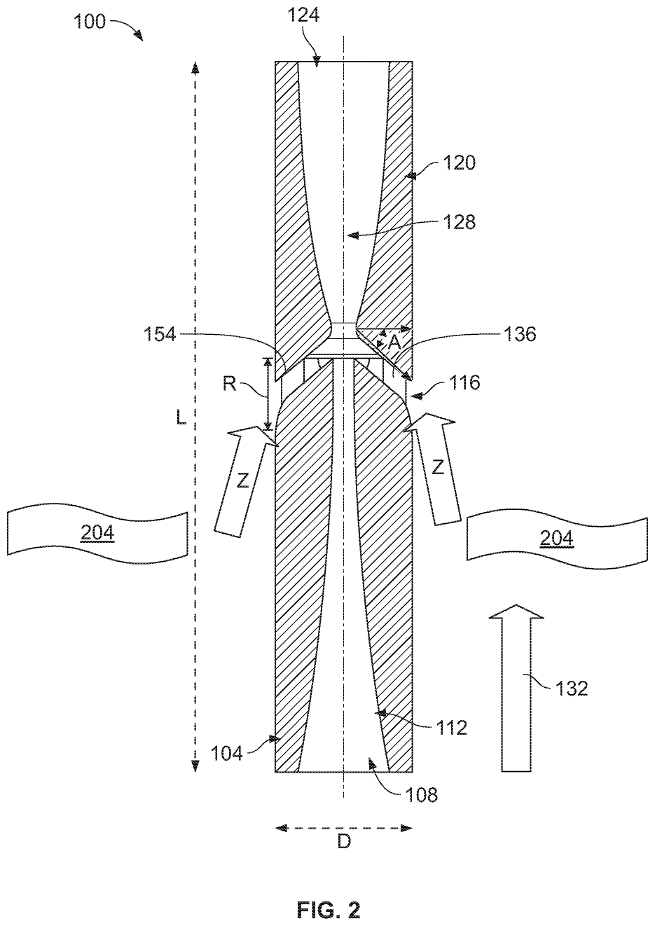

Referring still to FIG. 1, second inlet 116 may be formed from one or more angled surfaces (shown as 154 in FIG. 2) that form an angle relative to a longitudinal axis of vessel pump 100. In some embodiments, one or more angled surface of second inlet 116 may form an acute angle (shown as angle “A” in FIG. 2) relative to a longitudinal axis of vessel pump 100. An “acute” angle as used in this disclosure is an angle that is about or less than 90 degrees. “About” as used in this disclosure refers to a percentage different of two values anywhere from 0% to 20%. An angle between second inlet 116 and a wall of a vessel that vessel pump 100 may be positioned in may be obtuse relative to a central axis of vessel pump 100. A central axis may be an axis spanning across a diameter of vessel pump 100 at a mid section of vessel pump 100. “Obtuse” as used in this disclosure refers to an angle that is between about 90 degrees an and about 180 degrees. For instance, an angle between second inlet 116 and a central axis of vessel pump 100 may be about, but is not limited to, about 90 degrees to about 120 degrees, about 120 degrees to about 140 degrees, about 140 degrees to about 160 degrees, or about 160 degrees to about 180 degrees. Angled surfaces of second inlet 116 may form a gap between first portion 104 and second portion 120. A gap formed by second inlet 116 may be sized to allow a flow of fluid into vessel pump 100 from an exterior of vessel pump 100. Various widths of openings of second inlet 116 may allow for various ranges of fluid flow rates of a surrounding fluid into vessel pump 100. Widths of second inlet 116 may range from, but not limited to, about 0 mm to about 1 mm, about 1 mm to about 2 mm, about 2 mm to about 3 mm, about 3 mm to about 4 mm, about 4 mm to about 5 mm, or greater than about 5 mm. In some embodiments, a size of the gap formed by second inlet 116 may be circumferentially uniform around vessel pump 100. In other embodiments, a size of the gap formed by second inlet 116 may differ circumferentially in width at various points around vessel pump 100. Different sizes of gaps may be formed through adjustment of connecting structure 136. For instance, fluid may be present proximate one or more walls 150 of vessel pump 100. A flow of fluid in first fluid channel 112 may cause a drop in pressure near second inlet 116, which may create a vacuum effect, allowing for fluid of a higher pressure to flow to a lower pressure within vessel pump 100. A pressure difference between vessel pump 100 and a surrounding fluid may be about, but is not limited to, about 1 mmHg to about 10 mmHg, about 10 mmHg to about 20 mmHg, about 20 mmHg to about 30 mmHg, about 30 mmHg to about 40 mmHg, about 40 mmHg to about 50 mmHg, or greater than about 50 mmHg. In some embodiments, a pressure difference between vessel pump 100 and a surrounding fluid may be about, but is not limited to, about −1 mmHg to about −10 mmHg, about −10 mmHg to about −20 mmHg, about −20 mmHg to about −30 mmHg, about −30 mmHg to about −40 mmHg, about −40 mmHg to about −50 mmHg, or less than about −50 mmHg A surrounding fluid may enter into a junction between first fluid channel 112 and second fluid channel 128. An angle of second inlet 116 may align with a flow of fluid, such as in direction 132, which may allow for a substantially uninterrupted flow of fluid to occur through vessel pump 100. “Substantially uninterrupted” as used in this disclosure refers to a flow of fluid that has a reduced fluid flow rate of less than about 20% compare to a starting fluid flow rate. For instance, surrounding fluid may flow into vessel pump 100 via second inlet 116 and may mix with fluid flowing within first fluid channel 112. In some embodiments, a first fluid may flow through fluid channel 112 towards second inlet 116. A first fluid may be any fluid described herein, such as, but not limited to, blood, urine, bile, or other fluid. A second fluid may be a fluid surrounding vessel pump 100. A surrounding fluid may be an unwanted build up of fluid, such as, but not limited to, cerebral spinal fluids (CSP), edema, ascites fluid, or other forms of fluid build up. A first fluid may mix with a second fluid at a junction connecting first fluid channel 112 and second inlet 116.

With continued reference to FIG. 1, second portion 120 may have second fluid channel 128. Second fluid channel 128 may be cylindrical, tubular, substantially cylindrical, conical, or other shapes. Second fluid channel 128 may be tapered. For instance, second fluid channel 128 may increase in radius from connecting structure 136 towards output end 124. In some embodiments, connecting structure 136 may connect first portion 104 and second portion 120 such that first fluidic channel 112 and second fluidic channel 128 form a singular channel. A singular channel formed by first fluidic channel 112 and second fluidic channel 128 may be in an hourglass shape. In some embodiments, a tapering of second fluidic channel 128 may be designed to increase a volume of a flow of fluid, such as by decreasing an acceleration of a flow of fluid. For instance, in fluid channel 112, a volume of a first fluid may be about, but is not limited to, about 0.1 mL to about 1 mL, about 1 mL to about 5 mL, about 5 mL to about 10 mL, or greater than about 10 mL. A volume of a first fluid flowing within first fluidic channel 112 may decrease by about, but not limited to, about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 200%, or greater than about 200%. Second fluidic channel 128 may be formed to maintain a relatively constant flow rate between first inlet 108 and output end 120. A “relatively constant flow rate” as used in this discourse refers to a flow rate within 20% of an original flow rate. For instance, a flow rate at first inlet 108 may be about 100 mL/s to about 300 mL/s, while a flow rate of output end 124 may be about 100 mL/s to about 300 mL/s. A flow rate of output end 124 may be higher than a flow rate at first inlet 108, by about, but not limited to, about 1% to about 5%, about 5% to about 10%, about 10% to about 15%, about 15% to about 20%, or greater than about 20%. In some embodiments, a first fluid and a second fluid may be combined at junction between first fluidic channel 112 and second inlet 116. A combined fluid of a first fluid and a second fluid may flow through second fluidic channel 128 towards output end 124 in direction 132. In some embodiments, a combined fluid exiting output end 124 may have a velocity that may be the same or about the same as a velocity of a first fluid entering first portion 104. For instance, a velocity may be about, but is not limited to, about 1 cm/s to about 20 cm/s. Output end 124 may be a hole of second portion 120 that may allow for a fluidic output. Output end 124 may be circular, ovular, or other shapes. Output end 124 may have a radius equal to a radius of first inlet 108. In other embodiments, radii of first inlet 108 and output end 124 may differ. A combined fluid output that may leave output end 124 may enter a vessel that vessel pump 100 may be positioned in.

As a non-limiting example, vessel pump 100 may be positioned within a vein proximate ascites fluid. Ascites fluid may mix with blood via second inlet 116 and may exit vessel pump 100 at output end 124. A flow rate within the vein may be substantially unchanged, such as about a 20% or less change in an original flow rate of the vein.

Referring now to FIG. 2, a top-down view of the bottom half of vessel pump 100 as illustrated in FIG. 1 is shown. Vessel pump 100 may have first portion 104, first inlet 108, first fluidic channel 112, second inlet 116, connecting structure 136, second portion 120, second fluidic channel 128, and/or output end 124. The similarly referenced structures described above with reference to FIG. 1 may be similarly applied here in FIG. 2.

Vessel pump 100 may have a length L of about 1 cm to about 10 cm or greater. Vessel pump 100 may have a diameter of about 0.5 cm to about 5 cm or greater. A gap formed by second inlet 116 may be about 1 mm to about 5 mm in width. In some embodiments, second inlet 116 may be formed by a combining of an end of first portion 104 with an end of second portion 120 via connecting structure 136. An end of first portion 104 closest to second portion 120 may be angled, and an end of second portion 120 closest to first portion 104 may be angled. For instance an end of first portion 104 may be curved inwards towards a longitudinal axis of vessel pump 100 while an end of second portion 120 may be curved outwards with respect to a longitudinal axis of vessel pump 100. The first portion 104 and second portion 120 may form angled surfaces that may form second inlet 116. In some embodiments, vessel pump 100 may be positioned within a vessel that may be adjacent surrounding fluid 204. Surrounding fluid 204 may be any fluid described herein, without limitation. In an embodiment, surrounding fluid 204 may be ascites fluid. A fluid may flow through first inlet 108 into first fluidic channel 112. A tapering of first fluidic channel 112 may cause an acceleration and a drop in pressure of a flow of fluid within first fluidic channel 112. For instance, a first fluid entering fluidic channel 112 may experience an acceleration of about 10 cm/s² to about 20 cm/s², about 20 cm/s² to about 30 cm/s², about 30 cm/s² to about 40 cm/s², about 40 cm/s² to about 50 cm/s², about 50 cm/s² to about 60 cm/s², about 60 cm/s² to about 70 cm/s², about 70 cm/s² to about 80 cm/s², about 80 cm/s² to about 90 cm/s², or about 90 cm/s² about 100 cm/s². A drop in pressure may cause a pressure differential at second inlet 116 between an interior of vessel pump 100 and pressure of surrounding fluid 204. Surrounding fluid 204 may be pulled into vessel pump 100 due to a pressure differential, which may cause surrounding fluid 204 to mix with a fluid flowing in first fluidic channel 112, forming a combined fluid that may travel within second fluidic channel 128. Surrounding fluid 204 may enter vessel pump 100 via second inlet 116 in direction Z. In some embodiments, surrounding fluid 204 may enter vessel pump 100 through second inlet 116 in a linear direction. For instance, surround fluid 204 may enter into vessel pump 100 through second inlet 116 in a substantially straight line from any acute angle with respect to a longitudinal axis of vessel pump 100. A “substantially straight line” is a line that has between 0 degrees to about 10 degrees of offset in its travel. A gap or opening of second inlet 116 may be sized to avoid or minimize the formation of eddy currents. An “eddy current” as used in this disclosure refers to a swirling of fluid. A gap or opening of second inlet 116 may reduce eddy current formation within a vessel by about, but not limited to, about 50% to about 60%, about 60% to about 70%, about 70% to about 80%, about 80% to about 90%, or greater than about 90%. For instance, eddy currents may form proximate second inlet 116 due to a flow of surrounding fluid 204 into second inlet 116. Second fluid channel 128 may be tapered to increase in radius from second inlet 116 to output end 124. An increase in radius of second fluidic channel 128 may cause a deceleration of a combined fluid which may allow for an increased volume of fluid to travel within second fluidic channel 128. For instance, a deceleration of a combined fluid traveling from second inlet 116 towards second fluidic channel 128 may be about, but is not limited to, about −10 cm/s² to about −20 cm/s², about −20 cm/s² to about −30 cm/s², about −30 cm/s² to about −40 cm/s², about −40 cm/s² to about −50 cm/s², about −50 cm/s² to about −60 cm/s², about −60 cm/s² to about −70 cm/s², about −70 cm/s² to about −80 cm/s², about −80 cm/s² to about −90 cm/s², or about −90 cm/s² about −100 cm/s². A combined fluid flowing through second fluidic channel 128 may have a volume of but is not limited to, about 0.1 mL to about 1 mL, about 1 mL to about 5 mL, about 5 mL to about 10 mL, or greater than about 10 mL. A volume of a combined fluid flowing within second fluidic channel 112 may increase by about, but not limited to, about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to about 200%, or greater than about 200% compared to a volume of combined fluid at a junction between second inlet 116 and first fluidic channel 112. In some embodiments, a flow rate of a combined fluid exiting output end 124 may be about the same as a flow rate of fluid entering first inlet 108. In other embodiments, a flow rate of fluid entering first inlet 108 may be greater or less than a flow rate of a combined fluid exiting output end 124.

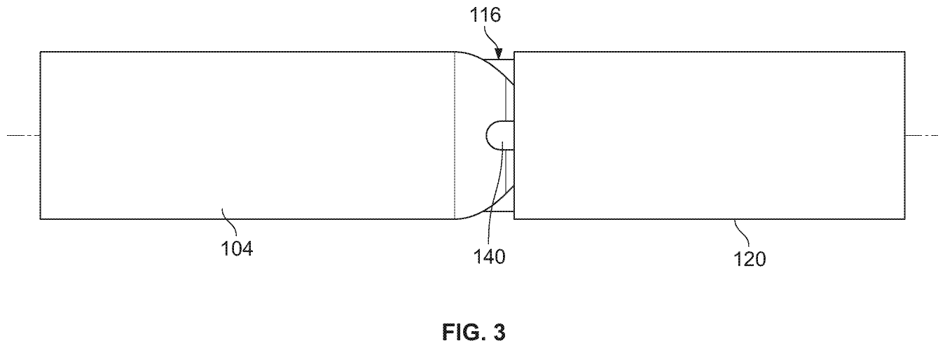

Referring now to FIG. 3, a side profile view of vessel pump 100 is presented. Vessel pump 100 may include first portion 104, second portion 120, and connecting structure 136, as described above with reference to FIGS. 1-2. Connecting structure 136 may include one or more legs 140. Legs 140 may protrude from first portion 104 towards second portion 120. In some embodiments, vessel pump 100 may have at least three legs 140. In embodiments where vessel pump 100 has at least three legs 140, each leg may have a corresponding X-axis, Y-axis, and Z-axis location with respect to an XYZ plane. A first leg may be have a greater positive X-axis location than one or more other legs, a second leg may have a greater positive Y-axis location than one or more other legs, and a third leg may have a negative X-axis location less than one or more other legs. Each leg in a three-leg system may have a positive or negative Z-axis location Multiple legs 140 may provide structural support between first portion 104 and second portion 120 against forces in an X-axis direction, Y-axis direction, and/or Z-axis direction. For instance, one or more legs 140 may resist a force in an X, Y, and/or Z-axis direction. Legs 140 may be circular, rectangular, ovular, or other shapes. In some embodiments, second inlet 116 may be part of connecting structure 136. For instance, second inlet 116 may be made of empty spaces surrounding one or more legs 140, which may allow surrounding fluid to enter into vessel pump 100. A length of one or more legs 140 may correspond linearly to a width of an opening of second inlet 116. For instance, a greater length of one or more legs 140 may correspond to a greater width of an opening of second inlet 116. Likewise, a lesser length of one or more legs 140 may correspond to a lesser width of an opening of second inlet 116. Legs 140 may be threaded and may increase or decrease in length in response to a clockwise or counter clockwise rotation within a threaded hole of first portion 104 and/or second portion 120.

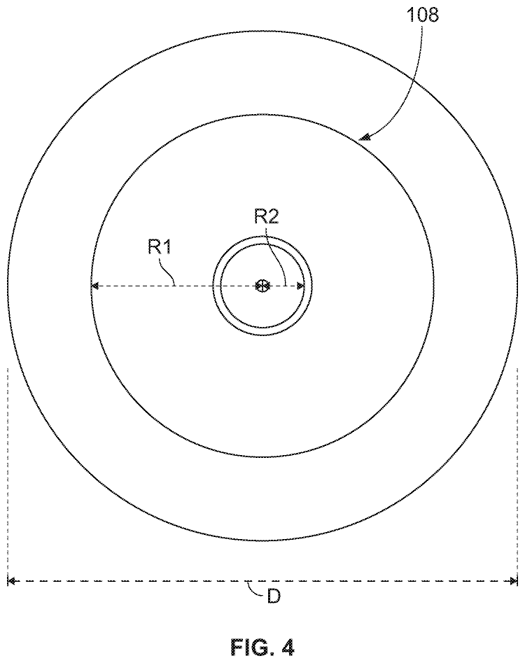

Referring now to FIG. 4, a front perspective view of vessel pump 100 is presented. Vessel pump 100 may include first inlet 108. First inlet 108 may be a conical or other shape. In some embodiments, first inlet 108 may have a gradually decreasing radius along a longitudinal axis of vessel pump 100. For instance, first inlet 108 may initially have a first radius and may taper inwards to a second radius, the second radius being less than the first radius. For instance, first inlet 108 may have first radius R1, which may be about, but is not limited to, about 0.5 mm to about 1 mm, about 1 mm to about 1.5 mm, about 1.5 mm to about 2 mm, about 2 mm to about 2.5 mm, about 2.5 mm to about 3 mm, or greater than about 3 mm. Second radius R2 may be about, but is not limited to, about 0.1 mm to about 0.2 mm, about 0.2 mm to about 0.3 mm, about 0.3 mm to about 0.4 mm, about 0.4 mm to about 0.5 mm, about 0.5 mm to about 0.6 mm, about 0.6 mm to about 0.7 mm, about 0.7 mm to about 0.8 mm, about 0.8 mm to about 0.9 mm, about 0.9 mm to about 1 mm, or greater than about 1 mm. In some embodiments, R1 may be about 1 cm to about 2 cm. R2 may be about 0.1 cm to about 1 cm. In some embodiments, R1 and/or R2 may be about 1 mm to about 12 mm. A tapering of first inlet 108 from a first radius to a second radius may allow for an acceleration of fluid flow through a first fluidic channel of vessel pump 100. Vessel pump 100 may have a circumference that may be sized to fit into one or more vessels. For instance, circumference of vessel pump 100 may be about 1*71 mm to about 20*71 mm.

### Deployable Vessel Pump

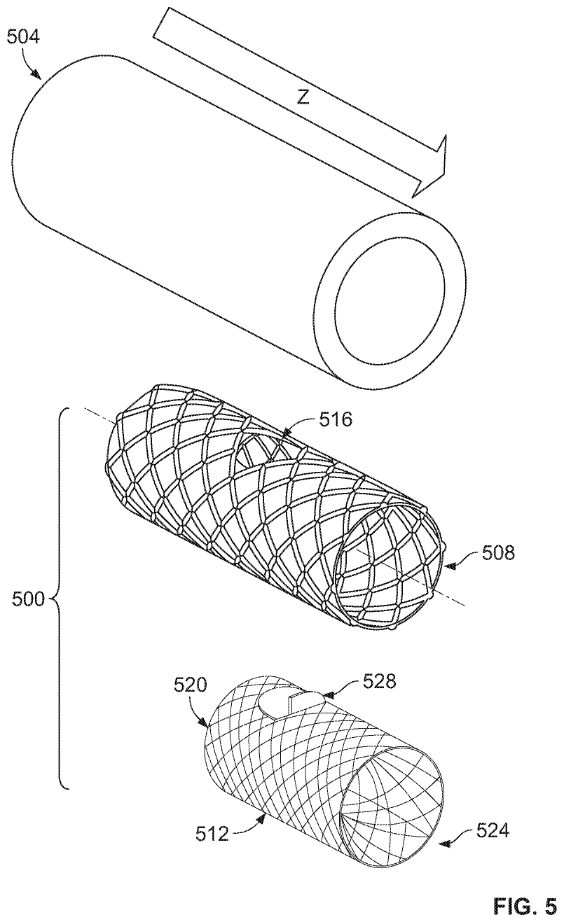

Referring now to FIG. 5, an exploded view of deployable vessel pump 500 and adjacent vessel 504 is shown. Vessel 504 may be a blood vessel or other vessel. Deployable vessel pump 500 may include graft 508 and tubular structure 512. A “tubular structure” as used in this disclosure refers to a substantially cylindrically shaped object. A “graft” as used in this disclosure refers to a structure that provides structural support to a part of the human body. Graft 508 may be made of, but is not limited to, Dacron, Polyethylene terephthalate (PET, expanded polytetrafluoroethylene (ePTFE), or other materials. Graft 508 may be a vascular or other graft. Graft 508 may have a radius of about, but not limited to, about 1 mm to about 2 mm, about 2 mm to about 3 mm, about 3 mm to about 4 mm, about 4 mm to about 5 mm, or greater than about 5 mm. Graft 508 may provide structural support to vessel 504. For instance, graft 508 may be inserted into vessel 504 and may assist in keeping vessel 504 expanded to a certain diameter and/or radius. Certain radii may include, but are not limited to, about 1 mm to about 2 mm, about 2 mm to about 3 mm, about 3 mm to about 4 mm, about 4 mm to about 5 mm, or greater than about 5 mm. In some embodiments, graft 504 may be made of a bioabsorbable material. A “bioabsorbable’ material as used in this disclosure refers to a property of a compound to dissolve within the human body. Bioabsorbable materials may include, but are not limited to, polymers, or other types of bioabsorbable materials. For instance, graft 508 may become absorbed into the human body after a period of time. A period of time may be, but is not limited to, hours, days, weeks, months, or years.

In some embodiments, graft 508 has opening 516. Opening 516 may be circular, ovular, or other shapes. Opening 516 may be positioned on a wall of graft 508. Opening 516 may have a radius of about, but not limited to, about 0.1 mm to about 0.2 mm, about 0.2 mm to about 0.3 mm, about 0.3 mm to about 0.4 mm, about 0.4 mm to about 0.5 mm, or greater than about 0.5 mm. Opening 516 may have a radius of about 0.1 mm to about 10 mm. Opening 516 may be formed at a center along a longitudinal axis of graft 504. In other embodiments, opening 516 may be formed closer to a proximal end of graft 508 or to a distal end of graft 508. In some embodiments, graft 508 may have a plurality of openings 516. Each opening 516 in a plurality of openings 516 may correspond to a fluidic inlet 528 of tubular structure 512. Opening 516 may be formed prior to graft 508 being inserted into vessel 504. In other embodiments, opening 516 may be formed while graft 508 is inserted into vessel 504. For instance, a sharp tool may be inserted into graft 504 and may rupture a portion of a wall of graft 504, which may form opening 516. Opening 516 may be positioned on a portion of a wall of graft 504 that may correspond to a position of a wall of vessel 504 that may be proximate a surrounding fluid. Graft 508 may be rotated to align with an area of interest of vessel 504 during or after insertion of graft 508 into vessel 504. An area of interest of vessel 504 may be a location along a wall of vessel 504 that may be proximate a fluid surrounding vessel 504, such as ascites fluid or any other fluid described herein. Opening 516 may be sized to allow fluidic inlet 520 of tubular structure 512 to come into contact with a fluid surrounding vessel 504.

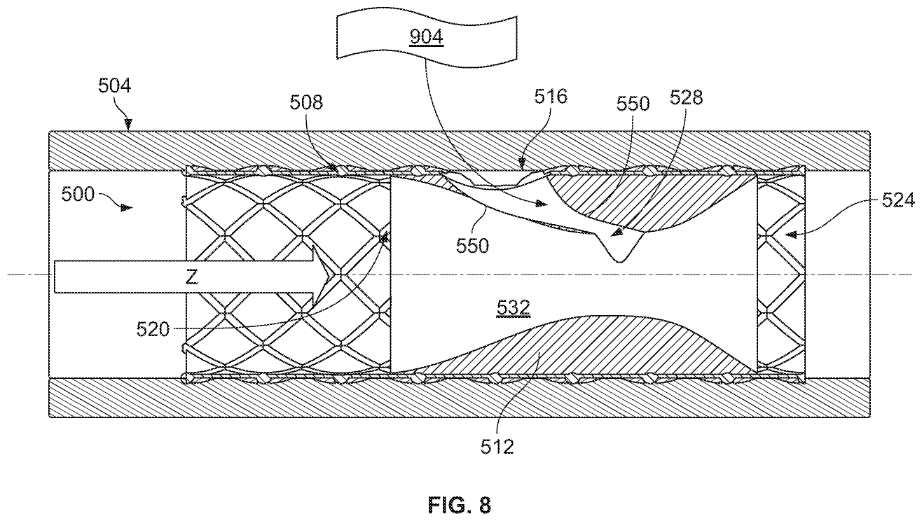

Tubular structure 512 may have a cylindrical outer structure and an hourglass shaped inner structure, as described in more detail below with reference to FIGS. 8A-B. In some embodiments, tubular structure 512 may be formed as a singular piece. For instance, tubular structure 512 may be mold manufactured with fluidic inlet 528. A cylindrical outer structure of tubular structure 512 may be reflective of a tubular geometry of vessel 504. For instance, a geometry of a cylindrical outer surface of tubular structure 512 may align with a geometry of a tubular structure of vessel 504, which may allow tubular structure 512 to be positioned within vessel 504 and/or graft 508. Tubular structure 512 may have first end 520 and second end 524, and a fluidic channel formed therebetween. A flow of a first fluid, such as, but not limited to, blood, may occur in vessel 504 in direction Z. First end 520 may direct a flow of a first fluid into a fluidic channel of tubular structure 512. A fluidic channel of tubular structure 512 may be hourglass shaped. An hourglass shaping of a fluidic channel of tubular structure 512 may cause a flow of a first fluid within tubular structure 512 to accelerate as a radius of the fluid channel decreases towards a center point of the fluidic channel at which the radius is at a minimum of the fluidic channel. An acceleration of a flow of a first fluid may cause a decrease in pressure, which may cause a pressure differential between a first fluid and a second fluid. A second fluid may be a fluid surrounding vessel 504, such as, but not limited to, ascites fluid. A pressure differential may cause a second fluid to be directed into a fluidic channel of tubular structure 512 via fluidic inlet 528. For instance, fluidic inlet 528 may be positioned proximate a hole in a wall of vessel 504. A hole may be formed through inserting a sharp tool into graft 508 and punctuation a portion of vessel 504, which may allow for fluidic inlet 528 to become in contact with surrounding fluids of vessel 504. Fluidic inlet 528 may be formed of one or more angled surfaces that may be acute with respect to a longitudinal axis of tubular structure 512. Fluid inlet 526 may form a pathway from a wall of tubular structure 512 to a fluidic channel of tubular structure 512. A second fluid may be directed into a fluidic channel of tubular structure 512 and may be combined with a first fluid, which may be expelled from tubular structure at second end 528. Tubular structure 512 may function similarly to that of vessel pump 100 as described above with reference to FIGS. 1-4.

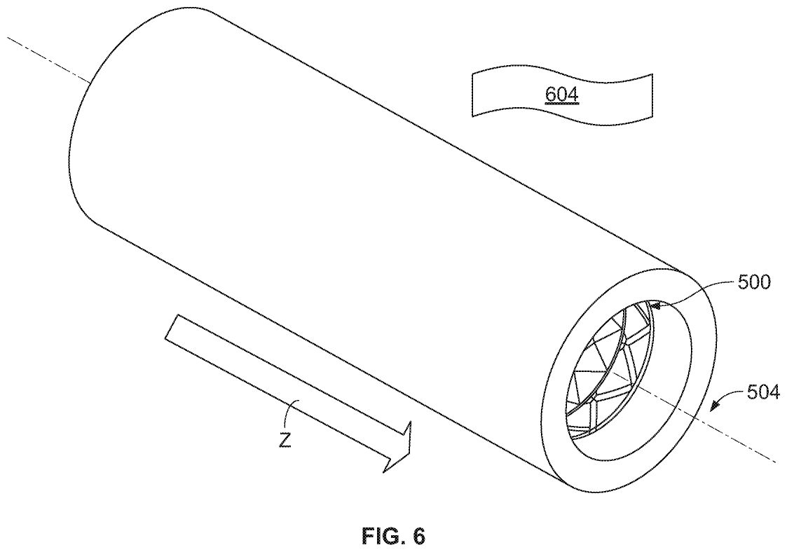

Referring now to FIG. 6, an illustration of the deployable vessel pump of FIG. 5 deployed inside vessel 504 is shown. Fluid may travel within vessel 504 and/or deployable vessel pump 500. Surrounding fluid 604 may be directed into deployable vessel pump 500 and may travel in direction Z along with a first flow of fluid, such as, but not limited to, blood. Deployable vessel pump 500 may be used to drain surrounding fluid 604 from an area proximate vessel 504. In some embodiments, surrounding fluid 604 may be proximate a left, right, bottom, and/or top side of vessel 504.

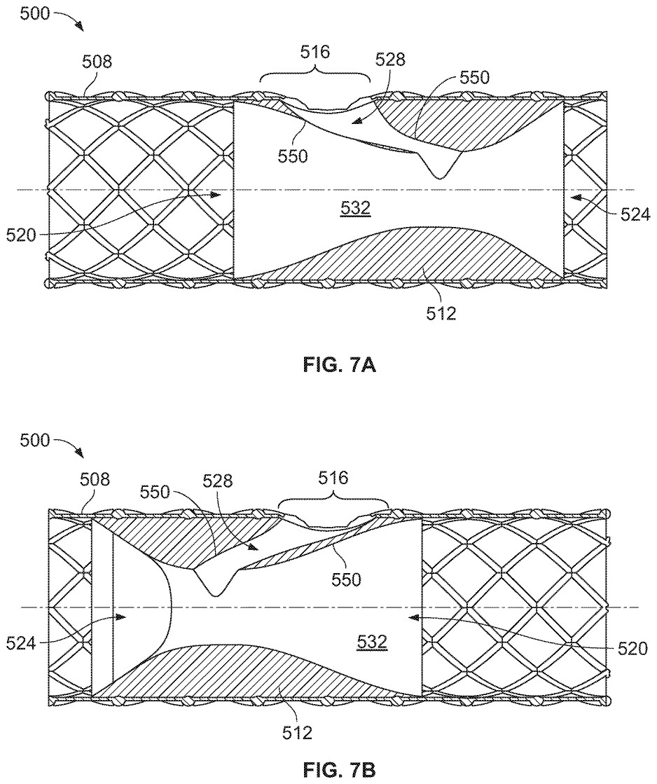

Referring now to FIG. 7A, a left side view of tubular structure 512 within graft 508 is presented. Tubular structure 512 may include first end 520, second end 524, and fluidic inlet 528. Graft 508 may have opening 516. Tubular structure 512 may have fluidic channel 532. Fluidic channel 532 may connect first end 520 to second end 524 and/or fluidic inlet 528. Fluidic channel 532 may be hourglass shaped. For instance, a radius of fluidic channel 532 may decrease from first end 524 towards fluidic inlet 528. A radius of fluidic channel 532 may increase from fluidic inlet 528 towards second end 524. In some embodiments, fluidic channel 532 may have an asymmetrical hour glass shape. For instance, a first portion of fluidic channel 532 extending towards fluidic inlet 528 from first end 520 may be larger than a second portion of fluidic channel 532 extending from fluidic inlet 528 towards second end 524. In some embodiments, a second portion of fluidic channel 532 may be larger than a first portion of fluidic channel 532.

Graft 508 may have opening 516. Opening 516 may allow for fluidic inlet 528 to become in fluidic communication with a fluid surrounding a vessel via a hole that may be formed in the vessel. Fluidic inlet 528 may be positioned on a portion of tubular structure 512, such as a top, bottom, left, or right portion with respect to a longitudinal axis of tubular structure 512. Fluidic inlet 528 may extend from an outermost wall of tubular structure 512 towards fluidic channel 532. In some embodiments, fluidic inlet 528 may be formed of two or more angled surfaces that may form a gap within tubular structure 512. Angled surfaces 550 of fluidic inlet 528 may have acute angles with respect to a longitudinal axis of tubular structure 512. Fluidic inlet 528 may be positioned at any point around a circumference of tubular structure 512. In some embodiments, tubular structure 512 may have two or more fluidic inlets 528. For instance, tubular structure 512 may have a first fluidic inlet and a second fluidic inlet positioned opposite the first fluidic inlet 528. Multiple fluidic inlets 528 may allow for a fluid intake of surrounding fluid at multiple points around a vessel. In some embodiments, fluidic inlet 528 may be at least partially circumferential with respect to tubular structure 512. For instance, fluidic inlet 528 may substantially circumferentially encompass tubular structure 512. “Substantially circumferentially” as used in this disclosure refers to an encompassing of an object that covers at least 90% of a circumference of an object. In embodiments where fluidic inlet 528 circumferentially encompass tubular structure 512, fluidic inlet 528 may direct a surrounding fluid at multiple angles relative to a longitudinal axis of a vessel towards fluidic channel 532.

Referring now to FIG. 7B, a right side view of deployable vessel pump 500 is presented. Deployable vessel pump 500 may include graft 504 and tubular structure 512, which may be as described above. In some embodiments, fluidic inlet 528 may be curved. For instance, fluidic inlet 528 may be curved towards a point of minimal radius of tubular structure 512 from a wall of tubular structure 512. In some embodiments, fluidic inlet 516 may form a fluidic pathway towards fluidic channel 532. A fluidic pathway of fluidic inlet 528 may have a constant diameter. In some embodiments, a fluidic pathway of fluidic inlet 528 may have a changing diameter. A diameter of a fluidic pathway of fluidic inlet 528 may decrease or increase from opening 516 towards fluidic channel 532. In some embodiments, a fluidic pathway of fluidic inlet 528 may have a cross-sectional area of about 0.5 mm² to about 5 mm². In some embodiments, a cross-sectional area of a fluidic pathway of fluidic inlet 528 may be constant. In other embodiments, a cross-sectional area of a fluidic pathway of fluidic inlet 528 may change with respect to a vertical axis of valve 500.

Deployable Vessel Pump within a Vessel

Referring now to FIG. 8, a side view of deployable vessel pump 500 in vessel 504 is presented. Vessel 504 may be a blood vessel or any other type of vessel. A first fluid may flow within vessel 504 towards deployable vessel pump 500. A first fluid may be blood or any other type of fluid described herein. A first flow of fluid may accelerate as it enters fluidic channel 532 through first end 520. For instance, a decrease in radius of fluidic channel 532 may cause an acceleration of a first fluid flowing in direction Z, which may result in a lower pressure of the first fluid. A pressure of a first fluid may be lowest at a minimal radius of fluidic channel 532. A decreased pressure of a first fluid may cause surrounding fluid 904 to have a higher pressure relative to the first fluid. A hole may be formed within a portion of vessel 504 which may allow surrounding fluid 904 to enter fluidic channel 532. For instance, a sharp tool may be inserted into graft 508 and may extend through or otherwise rupture a portion of vessel 504, such as a portion of vessel 504 proximate surrounding fluid 904. A pressure differential between surrounding fluid 904 and fluidic channel 532 may cause surrounding fluid 904 to enter fluidic channel 532 via fluidic inlet 528. Surrounding fluid 904 may combine with a first fluid to form a combined fluid which may exit tubular structure 512 through second end 524. Fluidic channel 532 may increase in radius from fluidic inlet 528 towards second end 524, which may cause a decrease in acceleration of a combined fluid flowing in direction Z within fluidic channel 532. A flow rate of a combined fluid flowing out of second end 524 may be about the same as that of a flow rate of a first fluid flowing in first end 520. For instance, a diameter and/or radius of first end 520 and second end 524 may be about the same. In some embodiments, a diameter and/or radius of first end 520 and second end 524 may be different.

### Additional Deployable Vessel Pump Embodiments

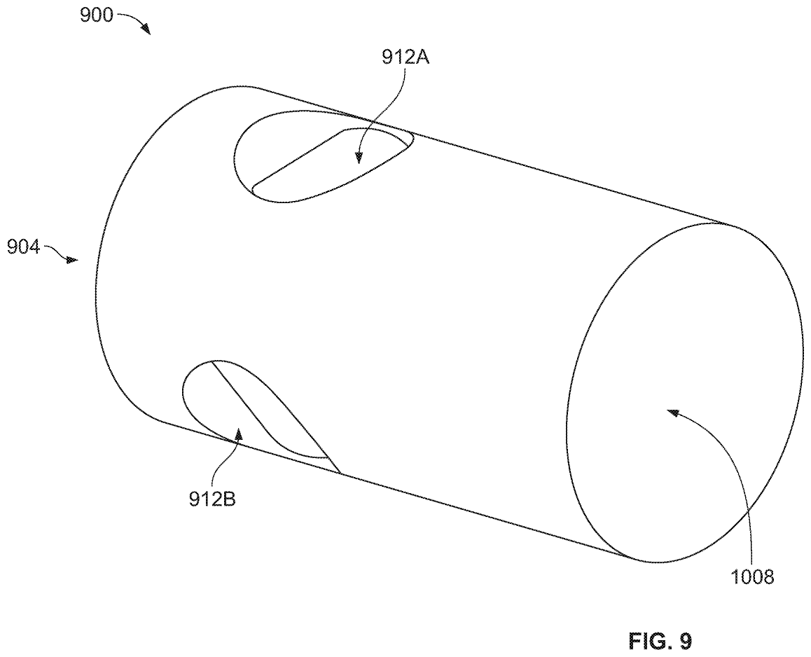

Referring now to FIG. 9, a side perspective view of deployable vessel pump 900 (“vessel pump 900”) is illustrated. Vessel pump 900 may be shaped as a cylindrical or tubular structure. In some embodiments, an outside surface of vessel pump 900 is shaped cylindrically. An outside surface of vessel pump 900 may be shaped to fit within a vessel or other tubular structure of the human body. In some embodiments, an interior of vessel pump 900 may have an hourglass shape. Vessel pump 900 may have first end 904. First end 904 may be an opening at an end of vessel pump 900 that may face an incoming flow of fluid. For instance, a flow of fluid originating from a vessel that vessel pump 900 may be positioned in may contact first end 904. First end 904 may direct and/or funnel a flow of a first fluid, such as, but not limited to, blood, into a fluidic channel of vessel pump 900. A fluidic channel of vessel pump 900 may be tapered. For instance, a fluidic channel of vessel pump 900 may decrease in radius along a longitudinal axis of vessel pump 900. A decrease in radius may cause a flow of a first fluid to experience an acceleration, which may cause a drop in pressure of the flow of the first fluid. A drop in pressure of a flow of a first fluid may cause a pressure differential between a fluidic channel of vessel pump 900 and a surrounding fluid of vessel pump 900, such as, but not limited to, ascites fluid, cerebral spinal fluid, bile, or any other fluid.

Vessel pump 900 may have or more fluidic inlets 912. For instance, vessel pump 900 may have first fluidic inlet 912A and second fluidic inlet 912B. Fluidic inlet 912A and/or 912B may be circular, cylindrical, or other shapes. Fluidic inlet 912A and/or 912B may be provide a fluidic pathway between a surrounding fluid of vessel pump 900 and a fluidic channel of an interior of vessel pump 900. A fluidic pathway of fluidic inlet 912A may extend from an exterior surface of vessel pump 900 towards a fluidic channel of an interior of vessel pump 900. A pressure differential may cause a surrounding fluid to flow into fluidic inlet 912A, which may direct the surrounding fluid into a fluidic channel of vessel pump 900. Likewise, a pressure differential may cause a surrounding fluid to flow into fluidic inlet 912B, which may direct the surrounding fluid into a fluidic channel of vessel pump 900. In some embodiments, vessel pump 900 may have three or more fluidic inlets 912. A surrounding fluid may be directed into a fluidic channel of vessel pump 900 and may combine with a flow of a first fluid to form a combined fluid. A combined fluid may exit out of second end 908 of vessel pump 900. Second end 908 may be shaped similarly or the same to that of first end 904. For instance, both first end 904 and second end 908 may be circular, cylindrical, or other shapes. A fluidic channel may increase in radius from a junction of first and/or second fluidic inlets 912A,B, towards second end 908. An increase in radius may cause a decrease in acceleration of a flow of fluid within a fluidic channel of vessel pump 900, which may increase a pressure of a combined fluid flowing towards second end 908. In some embodiments, a fluidic channel of vessel pump 900 may be formed such that a fluid flow rate entering first end 904 is about the same as a fluid flow rate of fluid exiting second end 908.

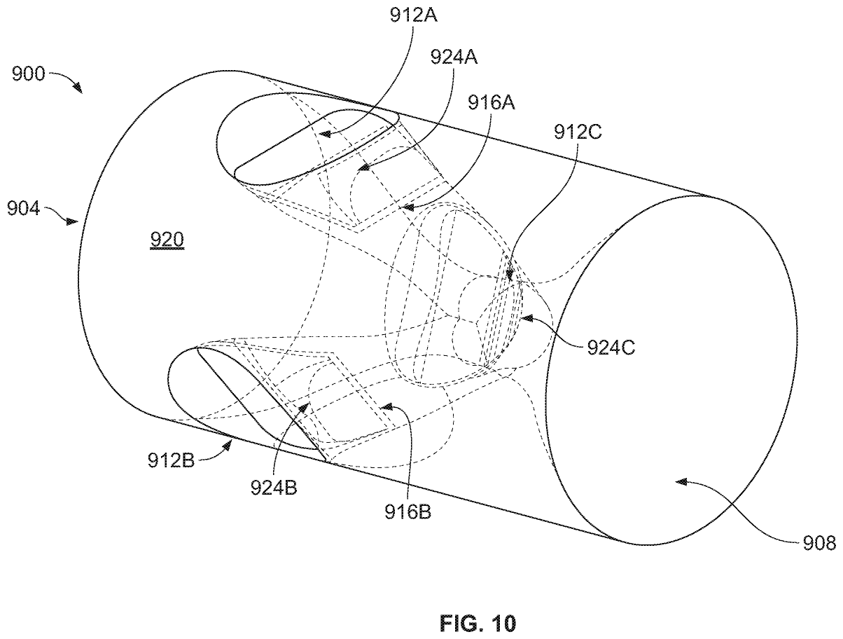

Referring now to FIG. 10, an X-ray view of vessel pump 900 is presented. Vessel pump 900 may have first end 904, second end 908, and fluidic inlets 912A, 912B, and/or 912C. In some embodiments, each fluidic inlet 912 may be separated by an angle of about 120 degrees, greater than about 120 degrees, or less than about 120 degrees with respect to a longitudinal axis of vessel pump 900. For instance, fluidic inlet 912A may be positioned at an angle of about 120 degrees from fluidic inlet 912C. Likewise, fluidic inlet 912A may be positioned at an angle of about 120 degrees from fluidic inlet 912B. Each fluidic inlet 912 may have a corresponding fluidic channel 916. For instance, fluidic inlet 912A may have fluidic channel 916A and/or fluidic inlet 912B may have fluidic channel 916B. Fluidic inlet 912C may have a fluidic channel. Fluidic channels 916 may connect a fluidic inlet 912 to fluid pathway 920 of vessel pump 900. Fluid pathway 920 may be formed from an interior surface of vessel pump 900. For instance, an exterior surface of vessel pump 900 may be shaped cylindrical, and an interior of vessel pump 900 may be shaped in an hourglass fashion. For instance, fluid pathway 920 may decrease in radius until a junction between one or more fluidic channels 916 and fluid pathway 920. At a junction between one or more fluidic channels 916 and fluid pathway 920, fluid pathway 920 may begin to increase in radius along a path towards second end 908.

In some embodiments, each fluidic inlet 912 may have a corresponding valve 924. For instance, fluidic inlet 912A may have valve 924A, fluidic inlet 912B may have valve 912B, and fluidic inlet 912C may have a valve. Valves 924 may be shaped as one-way valves. For instance, valves 924 may be designed in a duck-bill fashion, narrowing from fluidic inlet 912 towards fluidic channel 916, which may reduce an amount of fluid flowing from fluid pathway 920 and/or fluidic channels 916 towards fluid inlets 912. Valves 924 may help prevent eddy currents by directing surrounding fluid into fluidic channels 916 through fluidic inlets 912.

Referring now to FIG. 11, a front X-ray view of vessel pump 900 is presented. Vessel pump 900 may have fluidic inlets 912A, 912B, and/or 912C. Each fluidic inlet may have a corresponding fluidic channel 916A, 916B, and/or 916C. Each fluidic inlet 916 may be separated by angle Y. Angle Y may be about 120 degrees, greater than about 120 degrees, or less than about 120 degrees. In some embodiments, each fluidic inlet 916 may be separated by the an angle of equal value. In other embodiments, one or more fluidic inlets 916 may be separated by differing angles.

In some embodiments, vessel pump 900 has junction 950. Junction 950 may be a point in a fluid pathway of vessel pump 900 at which one or more fluidic channels 916 of one or more fluid inlets 912 connect with the fluid pathway. In some embodiments, junction 950 may be positioned at a longitudinal center of vessel pump 900. In some embodiments, fluidic inlets 912 and fluidic channels 916 may be symmetric across each side of a vertical axis of vessel pump 900. For instance, each fluidic inlet 912 may have a same width and/or length as one another. Each fluidic channel 916 may have a same radius, width, and tapering as each other.

Referring now to FIG. 12, a side view of an embodiment of vessel pump 900 is presented. Vessel pump 900 may have first end 904, second end 908, fluid pathway 920, fluidic inlet 912, fluidic channel 916, and/or valve 924. Fluid may may flow into fluid pathway 920 in direction Z. For instance, a first fluid originating from a vessel that vessel pump 900 may be positioned in may flow into fluid pathway 920 through first end 904. A flow of a first fluid in direction Z may accelerate as fluid pathway 920 decrease in radius, which may cause a drop in pressure of the flow of fluid in direction Z up to a junction point between fluidic channel 916 and fluid pathway 920. A drop in pressure may cause a surrounding fluid to enter into fluidic channel 916 via fluid inlet 912 in direction Y. Although direction Y is depicted as being linear, surrounding fluid may enter into fluidic inlet 912 from any angle along any path between fluid inlet 912 and fluidic channel 916. For instance, a pressure in a surrounding fluid may be higher than a pressure of a first fluid entering into fluid pathway 920. Fluid inlet 912 may have valve 924. Valve 924 may have a first angled surface and a second angled surface that may extend from one or more portions of fluidic inlet 912. An end of a first angled surface of valve 924 may contact an end of a second angled surface of valve 924, which may form a duck-bill shaped valve. Valve 924 may direct fluid flowing in direction Y in a one-way manner. For instance, valve 924 may open to allow a flow of fluid in direction Y when a pressure differential is created between fluid pathway 920 and a surrounding fluid. Valve 924 may close in a closed state when a pressure differential is not great enough to allow a flow of fluid in direction Y. A threshold pressure differential may be about, but is not limited to, about 1 mmHg to about 5 mmHg.

Referring now to FIG. 13A, an embodiment of vessel pump 900 is presented. Vessel pump 900 may have first end 904, second end 908, fluid pathway 920, first fluidic inlet 912A, and second fluidic inlet 912B. Fluidic inlet 912A may connect to fluid pathway 920 via first fluidic channel 916A. Second fluidic inlet 912B may connect to fluid pathway 920 through second fluidic channel 916B. First fluidic inlet 912A may direct a surrounding flow of fluid towards fluid pathway 920 in direction Y via fluidic channel 916A. For instance, a drop in pressure of a first fluid flowing in direction Z may cause a pressure differential between fluid pathway 920 and a surrounding fluid of vessel pump 900. Second fluidic channel 916B may direct a surrounding flow of fluid towards fluid pathway 920 in direction X towards fluid pathway 920 via second fluidic channel 916B. For instance, a drop in pressure of a first fluid flowing in direction Z may cause a pressure differential between fluid pathway 920 and a surrounding fluid of vessel pump 900. In some embodiments, a pressure differential between a surrounding fluid proximate first fluidic inlet 912A and a surrounding fluid proximate second fluidic inlet 912B may be different. For instance, a pressure differential at first fluidic inlet 912A may be greater than a pressure differential proximate second fluidic inlet 912B or vice versa. A higher pressure differential may cause a more rapid flow rate of a surrounding fluid into fluidic inlet 912A or 912B, which may cause an uneven amount of fluid to become joined at a junction between fluidic channel 916A and fluid pathway 920 and a junction between fluidic channel 916B and fluid pathway 920. In other embodiments, a same amount of fluid or flow rate may occur at both fluidic channel 916A and fluidic channel 916B. Although direction X is depicted as linear, a surrounding fluid may be directed into fluid pathway 920 in any angle along any curvature or line between second fluidic inlet 912B and fluid pathway 920. In some embodiments, a fluid flowing in direction Y may be a same fluid that may flow in direction X. In other embodiments, a fluid flowing in direction Y may be different than a fluid flowing in direction X. Fluids flowing in from first and second fluidic inlets 912A,B, may combine with a first fluid flowing from first end 904 in direction Z. A combined fluid may flow out of vessel pump 900 via second end 908. Fluid pathway 920 may expand in radius from a junction between first fluidic inlet 912A and/or second fluidic inlet 912B and fluid pathway 920 towards second end 908. An expanding radius of fluid pathway 920 may cause a deceleration of a combined fluid flowing in direction Z through second end 908.

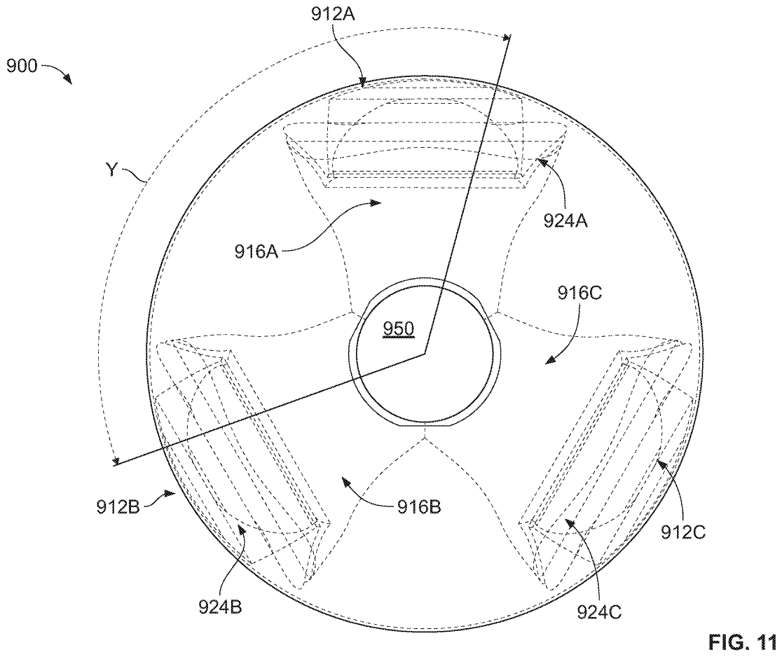

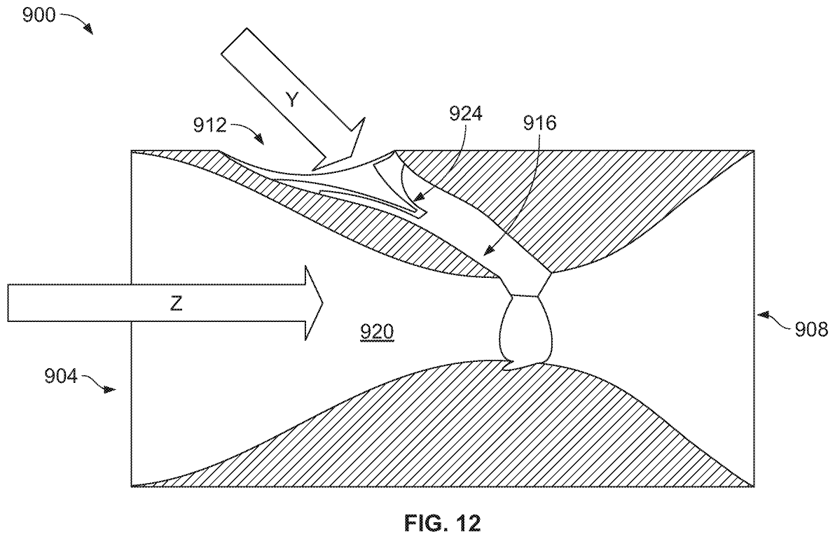

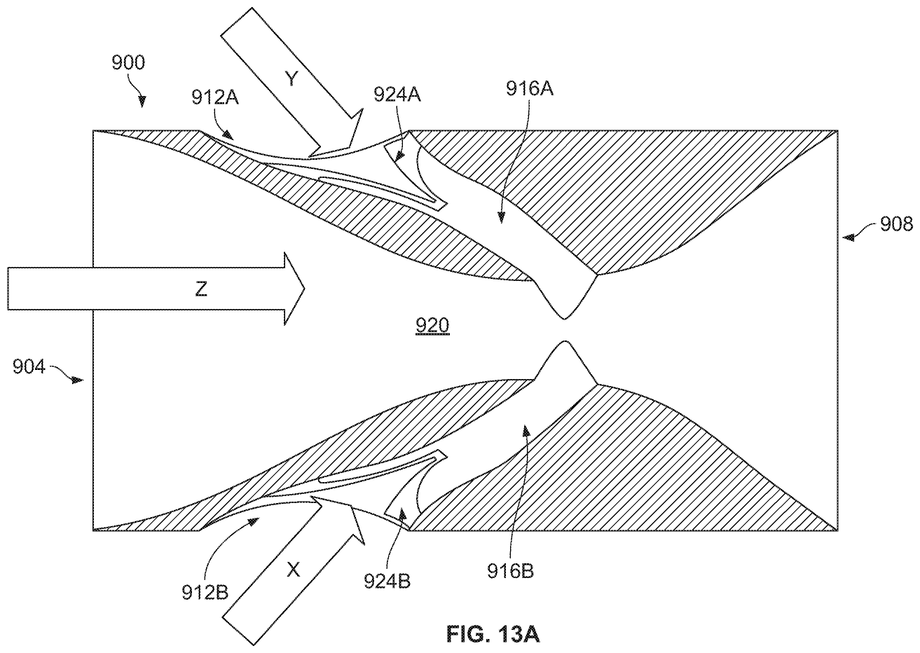

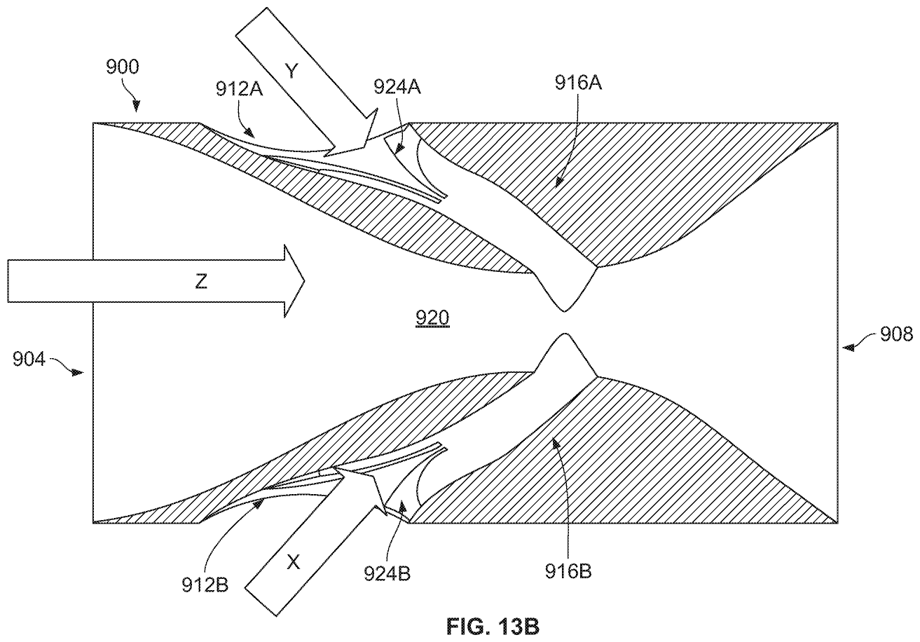

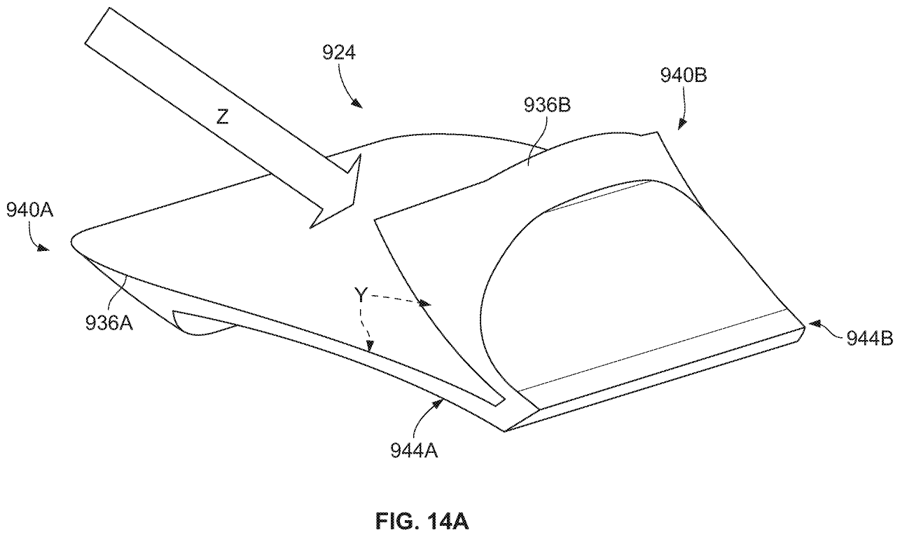

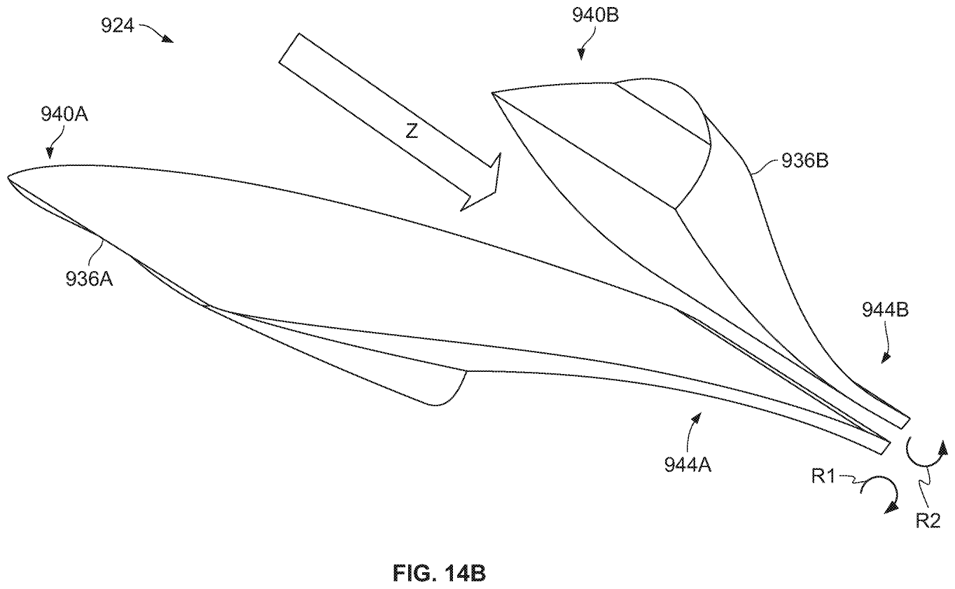

Referring now to FIG. 13B, vessel pump 900 with valves 924A and 924B in open states is illustrated. In some embodiments, both valves 924A and 924B are in open states. In some embodiments, valve 924A is in an open state while valve 924B is in a closed state or vice versa. A pressure experience by valve 924A may be about 1 mmHg to about 5 mmHg, which may cause valve 924A to open. One or more angled surfaces of valve 924A may rotate by about, but not limited to, about 0 degrees to about 5 degrees, about 5 degrees to about 10 degrees, about 10 degrees to about 15 degrees, about 15 degrees to about 20 degrees, about 20 degrees to about 25 degrees, about 25 degrees to about 30 degrees, about 30 degrees to about 35 degrees, about 35 degrees to about 40 degrees, about 40 degrees to about 45 degrees, or greater than about 45 degrees. A rotation of one or more angled surfaces of valve 924A may cause an opening between angled surfaces of 924A, which may allow for a flow of fluid in direction Y. Valve 924B may be as described with reference to valve 924A and may open, which may allow a flow of fluid in direction X. In some embodiments, a size of an opening created by valve 924A and/or 924B may correlate to a pressure experience by valve 924A and/or 924B. For instance and without limitation, a greater pressure experience by valves 924A,B, may cause a greater rotation of one or more angled surfaces of valves 924A,B, which may increase an opening between two or more angled surfaces of each of valves 924A,B. An increase in an opening between two or more angled surfaces of each of valves 924A,B, may allow for an increased amount of fluid entering into fluid pathway 920 from directions X and Y compared to a smaller sized opening created by two or more angled surfaces of valves 924A,B. In some embodiments, once a pressure differential threshold is reached, valves 924A,B may fully rotate open. A full rotation to fully open valves 924A,B may include rotating each angled surface of valves 924A,B, such that they contact one or more walls of fluidic inlets 912A,B. Operations of valves 924A,B, may be described in further detail below with reference to FIGS. 14A-14B.

Referring now to FIG. 14A, a perspective view of a valve in a closed state is presented. Valve 924 may be as described in FIG. 12. Valve 924 may be formed from two or more angled surfaces 936A,B. For instance, first angle surface 936A may be angled with respect to second angled surface 936B by angle Y. Angle Y may be an acute angle. For instance, angle Y may be about 90 degrees or less, about 80 degrees or less, about 70 degrees or less, about 60 degrees or less, about 50 degrees or less, about 40 degrees or less, about 30 degrees or less, about 20 degrees or less, or about 10 degrees or less. An angle between first angled surface 926A and second angled surface 936B may aid in directing a flow of a fluid surrounding within valve 924, such as in direction Z. In some embodiments, valve 924 may be formed in a duck-bill shape. For instance, first angled surface 936A may have first end 940A and second end 944A. First end 940A may be connected to a portion of a fluid inlet. In some embodiments, first end 940A may be rotatably connected to a portion of a fluid inlet. Second end 944A may be opposite first end 940A any may contact second end 944B of second angled surface 936B. Second end 944B of second angled surface 936B may be positioned opposite first end 940B of second angled surface 936B. First end 940B of second angled surface 936B may be connected to a portion of a fluid inlet. In some embodiments, first end 940B may be rotatably connected to a portion of a fluid inlet. First angled surface 936A may extend towards second angled surface 936B through second end 944A. Ends 944A and 944B of first angled surface 936A and second angled surface 936B may meet, which may prevent a flow of fluid entering valve 924 in direction Z. In some embodiments, first angled surface 936A and/or second angled surface 936B may be designed to remain in a closed state until a threshold pressure differential is experienced. A closed state may be a position in which first angled surface 936A and second angled surface 936B contact each other at ends 944A,B, which may reduce and/or preventing a flow of fluid in direction Z. A threshold pressure differential may be about, but is not limited to, about 1 mmHg to about 5 mmHg.

Referring now to FIG. 14B, valve 924 in an open state is illustrated. An open state may be a state in which first angled surface 936A and second angled surface 936B are rotated away from each other such that a flow of fluid may occur in direction Z. A pressure differential may be experienced by valve 924, which may cause first angled surface 936A and/or second angled surface 936B to rotate away from each other, which may allow a flow of fluid in direction Z through valve 924. Second end 944A may rotate in direction R1. Direction R1 may be clockwise. Second end 944B may rotate in direction R2. Direction R2 may be counter-clockwise. In some embodiments, first angled surface 936A and/or second angled surface 936B may rotate by about, but not limited to, about 0 degrees to about 10 degrees, about 10 degrees to about 20 degrees, about 20 degrees to about 30 degrees, about 30 degrees to about 40 degrees, about 40 degrees to about 50 degrees, about 50 degrees to about 60 degrees, about 60 degrees to about 70 degrees, about 70 degrees to about 80 degrees, about 80 degrees to about 90 degrees, or greater than about 90 degrees with respect to a longitudinal axis of valve 924. In some embodiments, a higher pressure differential experienced by valve 924 may cause a greater rotation of first angled surface 936A and/or second angled surface 936B. In other embodiments, at a threshold pressure differential, first angled surface 936A and/or second angled surface 936B may fully rotate away from each other. Fully rotating away from each other may include rotating Valve 924 may prevent a flow of fluid in a direction opposite direction Z. For instance, a flow of fluid in a direction opposite direction Z may lower a pressure differential experienced by valve 924, which may cause first angled surface 936A and/or second angled surface 936B to rotate towards each other, which may cause valve 924 to enter a closed state.

### Methods

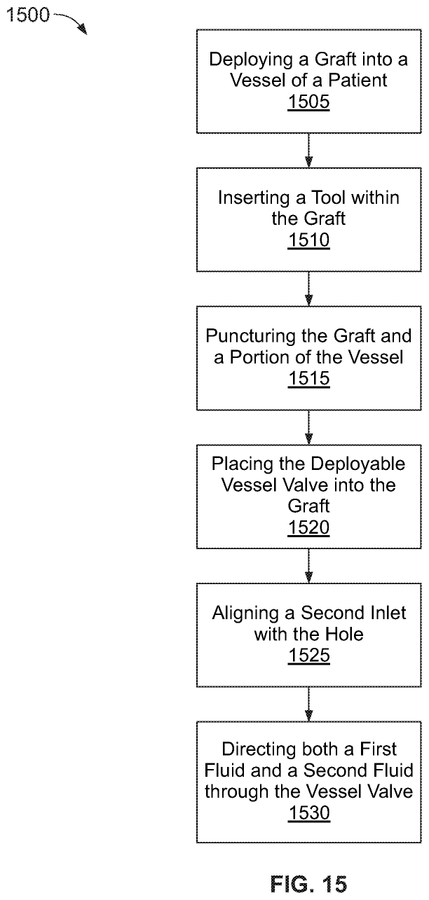

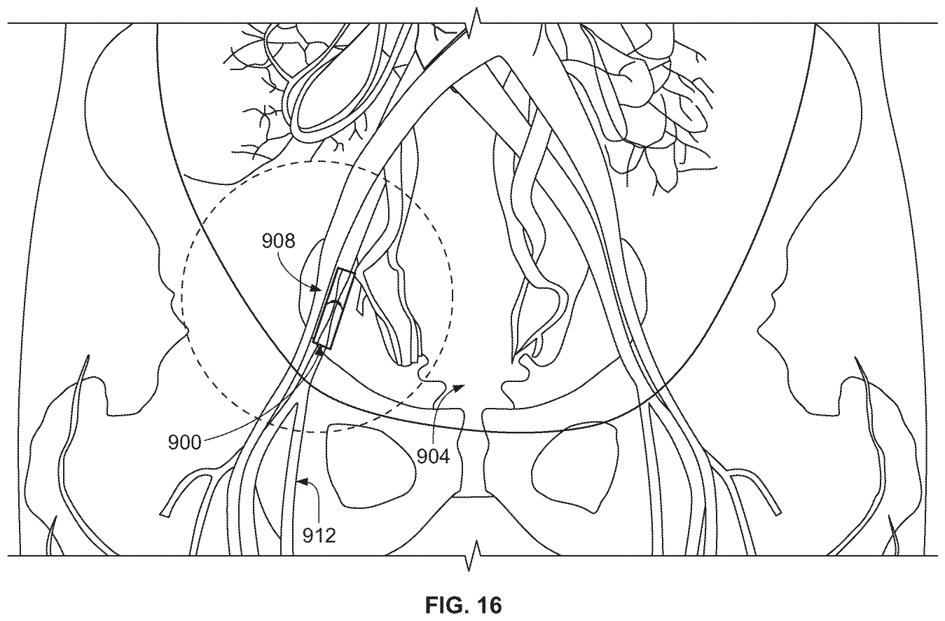

Referring now to FIG. 15, a flowchart illustrating a method 1500 of directing fluid within a patient using a deployable vessel pump is presented. At step 1505, method 1500 includes deploying a graft into a vessel of a patient. A graft may be a vascular graft, in some embodiments. A vessel of a patient may be a blood vessel, in some embodiments. In some embodiments, a balloon may be inserted into the graft. A ballon may be expanded within a graft which may cause a circumferential expansion of the graft. For instance, a balloon may be inflated, which may cause a circumferential expansion of a graft. A ballon may experience a pressure of about 1 atm to about 50 atm.

At step 1510, method 1500 includes inserting a tool within the graft. A tool may be a puncturing device that may have a sharp end designed to rupture or pierce a wall of a graft and/or vessel. A tool may have multiple sharp edges, in some embodiments.

At step 1515, method 1500 includes puncturing the graft and a portion of the vessel. The graft and/or the vessel may be punctured by a tool. In some embodiments, the graft and vessel may be punctured in a single motion by a tool. In some embodiments, two or more portions of the vessel may be punctured, which may form two or more holes within the vessel.

At step 1520, method 1500 includes placing the deployable vessel pump into the graft. A deployable vessel pump may be placed into a graft using one or more surgical tools. Surgical tools may include, but are not limited to, guidewires, catheters, and the like. A deployable vessel pump may have a first end, a second end, and a fluidic channel formed therebetween. A first end may have a first inlet having an opening designed to allow a flow of a first fluid originating from the vessel to flow into the fluidic channel through the first inlet.

At step 1525, method 1500 includes aligning a second inlet of a deployable vessel pump with a hole formed within a vessel. A second inlet may be in fluidic communication with a fluidic channel of a deployable vessel pump. A second inlet may have one or more angled surfaces that may form an acute angle relative to a longitudinal axis of a deployable vessel pump.

At step 1530, method 1500 includes directing both a first fluid and a second fluid through the vessel pump. A flow of a first fluid within a fluidic channel of a deployable vessel pump may cause a pressure differential between the first fluid and a second fluid surrounding a vessel. A pressure differential may cause a second fluid surrounding a vessel to be pulled into a second inlet and mixed with a first fluid flowing in a fluidic channel of a deployable vessel pump to form a combined fluid. A combined fluid may be output from a deployable vessel pump at a second end of the deployable vessel pump.

### Bernoulli's Principle

In some embodiments, devices, systems, and methods described herein may utilize Bernoulli's principle, which states that in a flowing fluid, an increase in the fluid's speed occurs simultaneously with a decrease in pressure or potential energy. Bernoulli's principle may be give by the following equation.

$$P + \frac{1}{2} ρ {v}^{2} + ρ gh = constant \qquad Equation 1$$

where P is the absolute pressure, p is the fluid density, v is the velocity of the fluid, h is the height above some reference point, and g is the acceleration due to gravity. As v increases, P decreases to keep the constant value constant. In other words, an increase in acceleration of a fluid causes a decrease in pressure of the fluid. Likewise, a deceleration of a fluid causes an increase in pressure of the fluid.

### Anatomical Examples

Referring now to FIG. 16, an illustration of a vessel pump within a vessel is presented. While FIG. 16 depicts the inferior Vena Cava, vessel pump 900 may be positioned within any vessel of the human body, without limitation. Vessel pump 900 may be placed within femoral vein 912 which may be adjacent external lliac artery 908. External lliac artery 908 may provide a main blood supply to the legs. External lliac artery 908 may pass down along the brim of the pelvis and may divide into two large branches, the inferior epigastric artery and the deep circumflex artery. Femoral vein 912 may return deoxygenated blood from the legs back to the heart. Femoral vein 912 may arise from the popliteal vein and may continue upwards through the thigh.

Fluid 904 may be proximate to either or both of femoral vein 912 and external iliac artery 908. Fluid 904 may be ascites fluid. Vessel pump 900 may be positioned within femoral vein 912. A tool may partially rupture a wall of femoral vein 912, which may allow a flow of fluid 904 into vessel pump 900. Vessel pump 900 may intake fluid 904 and combined it with fluid originating within femoral vein 912, such as blood. Femoral vein 912 may carry a combination of fluid 904 and blood throughout the body. For instance, a combination of fluid 904 and blood may flow to the heart and circulatory system of the body.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

1. A vessel pump positionable inside a vessel of a patient, comprising:
a first portion comprising a first inlet fluidically connected to a first fluidic channel, the first inlet comprising an opening allowing a first fluid originating from the vessel of the patient to flow through the opening into the first fluidic channel;
a second portion comprising a second fluidic channel fluidically connected to an output end;
a connecting structure connecting the first portion to the second portion, wherein the first fluidic channel and the second fluidic channel form a single fluidic channel when the first portion and the second portion are connected; and
a second inlet positioned at a junction between the first portion and the second portion, wherein the second inlet is in fluidic communication with the single fluidic channel through an angled surface that forms an acute angle relative to a longitudinal axis of the first portion, the second inlet having an opening width that, when the vessel pump is positioned inside the vessel, is sufficient to establish a pressure differential between a second fluid adjacent to the vessel and the first fluid flowing in the single fluidic channel, wherein the pressure differential causes at least a portion of the second fluid to be directed into the single fluidic channel through the second inlet.

2. The vessel pump of claim 1, wherein the first fluidic channel is tapered from the first portion towards the junction.

3. The vessel pump of claim 1, wherein the second fluidic channel is tapered from the junction towards the output end.

4. The vessel pump of claim 1, wherein the first fluid experiences an acceleration within the first fluid channel towards the junction.

5. The vessel pump of claim 1, wherein a velocity of the first fluid entering the first portion is the same as a velocity of the combined fluid leaving the second portion.

6. The vessel pump of claim 5, wherein the velocity is about 5 cm/s to about 50 cm/s.

7. The vessel pump of claim 1, wherein the connecting structure comprises at least three legs connecting the first portion to the second portion.

8. The vessel pump of claim 7, wherein the at least three legs provide a structural support to the vessel pump via in one or more of an X-axis, Y-axis, and Z-axis direction.

9. The vessel pump of claim 8, wherein the at least three legs are threaded.

10. The vessel pump of claim 9, wherein the at least three legs are adjustable in length, thereby causing an increase or decrease in width of the fluidic inlet.

11. The vessel pump of claim 1, wherein the fluid inlet is circumferential with respect to the vessel pump.

12. The vessel pump of claim 1, wherein the first fluidic channel is oriented within the vessel to be facing a direction of flow of the first fluid.

13. The vessel pump of claim 1, wherein the acute opening has a width of about 1 mm to about 5 mm.

14. The vessel pump of claim 1, wherein the first fluid is blood and the second fluid is ascites fluid.

15. The vessel pump of claim 1, wherein a pressure of the first fluid within the vessel pump is about 0 mmHg to about 120 mmHg and a pressure of the combined fluid within the vessel pump is about 0 mmHg to about 50 mmHg.

16. The vessel pump of claim 1, wherein the pressure of the second fluid pressure is about 0 mmHg to about 50 mmHg.

17. (canceled)

18. A deployable vessel pump, comprising:
a tubular structure comprising a first end, a second end, and a fluidic channel formed therebetween, wherein the first end comprises a first inlet comprising an opening allowing a flow of a first fluid of the vessel to into the fluidic channel through the opening;
a second inlet formed within the tubular structure, wherein the second inlet is in fluidic communication with the fluidic channel through an angled surface that forms an acute angle relative to a longitudinal axis of the tubular structure, the second inlet having an opening width that, when the deployable vessel pump is positioned inside the vessel, is sufficient to establish a pressure differential between a second fluid adjacent to the vessel and the first fluid flowing in the fluidic channel, wherein the pressure differential causes at least a portion of the second fluid to be directed into the fluidic channel through the second inlet; and
a graft circumferentially encompassing the tubular structure, wherein at least a portion of the graft is removed, thereby allowing a fluidic connection between the second fluid and the second inlet, forming a combined fluid.

19. The deployable vessel pump of claim 18, wherein the fluidic channel has an hourglass shape.

20.-56. (canceled)

57. A method of treating a patient with fluid buildup, comprising:
implanting a vessel pump into a vessel of the patient, the vessel pump comprising a first end, a second end, a fluidic channel formed therebetween, and a fluidic output end in fluid communication with the fluidic channel; and
aligning the fluidic output end with at least a hole of the vessel of the patient, wherein the fluidic inlet is in fluidic communication with the fluidic channel through an angled surface that forms an acute angle relative to a longitudinal axis of the vessel pump, the fluidic inlet having an opening width that, when the vessel pump is positioned inside the vessel, is sufficient to establish a pressure differential between a fluid adjacent to the vessel and a fluid flowing in the fluidic channel, wherein the pressure differential causes at least a portion of the fluid adjacent to the vessel to be directed into the fluidic channel through the second inlet.
